(19) United States
(12) Patent Application Publication
(10) Pub. No.: US 2009/0326539 A1
(43) Pub. Date: Dec. 31, 2009

(54) TREPAN DRILL

(75) Inventors: Stefan NEUMEYER, Eschlkam (DE); Michael KRUMSIEK, Lemgo (DE)

Correspondence Address: KOLISCH HARTWELL, P.C., 200 PACIFIC BUILDING, 520 SW YAMHILL STREET, PORTLAND, OR, 97204, US

(73) Assignee: GEBR. BRASSELER GMBH & CO. KG, Lemgo (DE)

(21) Appl. No.: 12/491,166

(22) Filed: Jun. 24, 2009

(30) Foreign Application Priority Data
Jun. 24, 2008 (DE) 10 2008 029 920.0

(51) Int. Cl. A61B 17/32

(52) U.S. Cl. 606/80

(57) ABSTRACT

A trepan drill that comprises a shaft that includes a clamping portion, and a head that comprises a substantially cylinder barrel shaped core bit and a distal end. The distal end includes a first tooth, a second tooth, and a third tooth that each respectively has a cutting edge. The teeth each have different axial teeth heights and are arranged alternatingly in a circumferential direction.

### CROSS-REFERENCE TO RELATED APPLICATION

This application claims priority to German Patent Application No. 10 2008 029 920.0, filed Jun. 24, 2008, which is hereby incorporated by reference.

### BACKGROUND

The present disclosure relates to a trepan drill.

For the implant care of jawbones, it may be required to obtain a bone cylinder from a patient's bone and to implant same at a suitable location. For this purpose, trepans are commonly used. For generating the hollow cylinder by use of trepans or trepan drills, it is required that same have continuous and good cutting characteristics and enable the extraction of an exact bone cylinder without thermally damaging the bone. The trepans known from the state of the art cannot adequately seize or bite into the bone, or may leave the exact course of the cutting, due to irregular pressure by the dentist or due to different force transmission through the bone to be processed. A fact that has been ignored up to now is that damages of the soft tissue by the trepan drill are possible. All these are undesired effects which adversely affect a quality bone management for the implant care.

### SUMMARY

A trepan drill that comprises a shaft that includes a clamping portion, and a head that comprises a substantially cylinder barrel shaped core bit and a distal end. The distal end includes a first tooth, a second tooth, and a third tooth that each respectively has a cutting edge. The teeth each have different axial teeth heights and are arranged alternatingly in a circumferential direction.

### BRIEF DESCRIPTION OF THE DRAWINGS

FIG. 1 shows a perspective view of a gingival punch.

FIG. 2 shows a perspective view of a centering trepan.

FIG. 3 shows a perspective view of a trepan drill.

FIG. 4 shows a detailed view of toothing of the trepan drill of FIG. 3.

FIG. 5 shows a partial plan view of the detailed view of FIG. 4.

FIG. 6 shows a view, analogous to FIG. 4, of another embodiment of the toothing.

FIG. 7-10 show different embodiments of recesses of a head of the trepan drill of FIG. 3.

FIG. 11 shows a view, similar to FIGS. 4 and 6, of toothing of the centering trepan of FIG. 2.

FIG. 12 shows a front view, analogous to FIG. 5, of the toothing shown in FIG. 11.

FIG. 13-16 respectively show partial sectional views of different embodiments of the centering trepan, along with a respectively enlarged front view of a centering element.

### DETAILED DESCRIPTION

In the different embodiments, identical parts are designated with identical reference numerals.

The trepan drill shown in FIG. 3 comprises a shaft 2 including a clamping portion 1. The clamping portion 1 is formed in the usual manner according to the state of the art and enables clamping and axially securing the trepan drill in a drive unit for transmitting the torque.

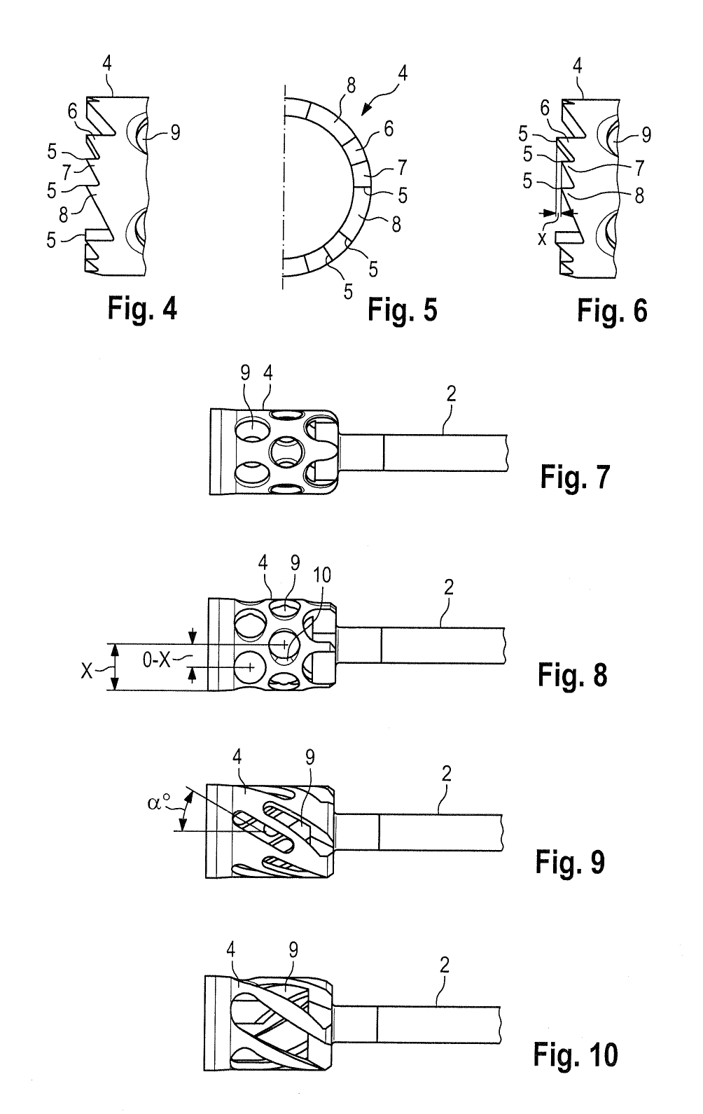

At the other end of the shaft 2, a head 3 is provided, which comprises a hollow cylinder or core bit 4 which is shaped like a cylinder barrel and is provided with teeth 6, 7, 8 at its distal end (see FIGS. 4 and 6). The teeth 6, 7, 8 may each respectively have at least one cutting edge 5.

The head 3 of the trepan drill is formed with a plurality of recesses 9, as is shown in particular in FIGS. 7 to 10. The recesses 9 serve to enhance the preparation (cleaning, sterilisation) as well as the chip reception, and further reduce the friction at the bone material such that damage to the bone material due to heat is minimized.

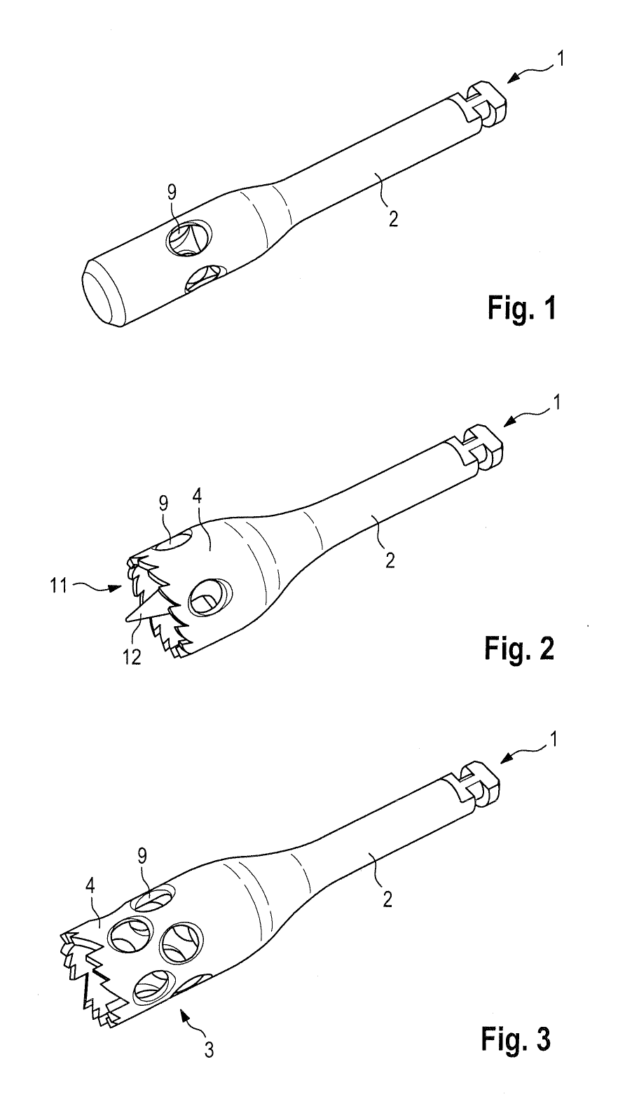

In a similar way, the centering trepan 11 according to FIG. 2 as well as the gingival punch according to FIG. 1 is provided with recesses 9.

In addition, the recesses 9 increase the torsional strength as well as the bending strength and offer a better visibility onto the region to be treated.

As is shown in FIGS. 7 to 10, the recesses 9 may be arranged and formed in different ways, e.g. as elongate slits (FIGS. 9 and 10) and as rows of round recesses (FIGS. 7 and 8). As is shown in FIG. 8, the axis of the recess 9 may be laterally offset by a certain amount with respect to the radial direction, such that a cutting edge 10 is formed, which has an aggressive cutting angle. The lateral offset is indicated by the amount 0-X, wherein 2*x corresponds to the diameter of the instrument. Contrary thereto, the round holes of the recesses 9 according to FIG. 7 are arranged radially with respect to the rotary axis of the trepan drill with regard to their center axis.

As shown in FIGS. 4 to 6, the head 3 comprises a group toothing including a first tooth 6 as well as two further teeth, i.e. a second tooth 7 and a third tooth 8. The first tooth 6 is larger in the axial direction than the second tooth 7 or the third tooth 8. Therewith, a plurality of group toothings of this kind result in the circumferential direction. In a varied embodiment according to FIG. 6, the second tooth 7 and the third tooth 8 are retreated in the axial direction, such that they have a tooth height being smaller by the amount x. Consequently, the first tooth 6 protrudes by the amount x. In this context, x corresponds maximally to the tooth depth of the deepest tooth.

It is apparent that also other combinations of such group toothings may be formed according to the present disclosure.

Altogether, the embodiment of the toothing results in a well controllable, smooth, and efficient operation of the trepan drill. Also in the head 3 of the trepan drill, it is possible to arrange the cutting edges 5 either radially or inclined by an angle γ with respect to the radial direction, as it is shown schematically in FIG. 12. The angle γ ranges between about −30° to about 30°. The cutting angle is preferably (but not always) formed in a range of about −20° to about +20°.

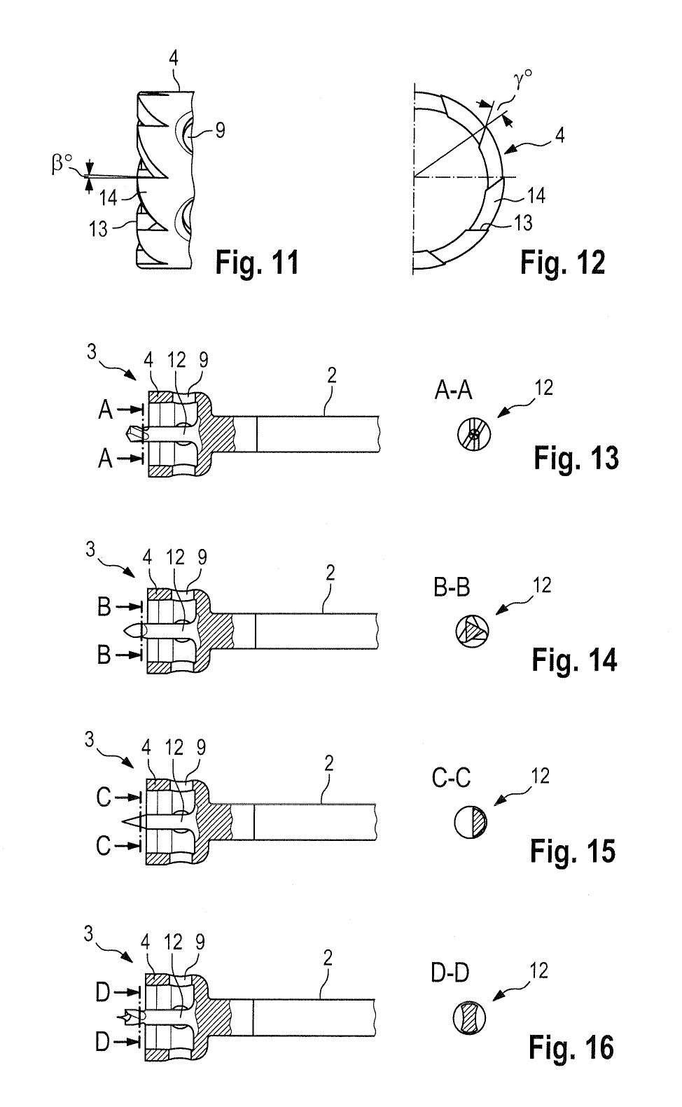

FIGS. 11 to 16 show the centering trepan according to the present disclosure. The head 3 thereof also comprises a hollow cylindrical core bit 4 which is provided with teeth 14 at its distal front face, which comprise cutting edges 13. As is shown in FIG. 12, the cutting edges 13 can also be arranged inclined by an angle y of about −30° to about 30° with respect to the radial direction.

The centering trepan 11 comprises an integrally formed centering element 12 which protrudes beyond the distal end of the core bit 4 (as is shown in FIGS. 13 to 16) and can be formed as a helical drill (FIG. 13), a triangle profile (FIG. 14), a single cutter (FIG. 15), or a wooden drill tip (FIG. 16).

Also in case of the centering trepan, the recesses 9 provide a reduced friction while offering an optimised visibility. By using the centering trepan, the extraction place of the bone cylinder may be defined exactly. An annular groove is produced, into which the trepan drill (FIG. 3) is subsequently inserted and can be guided for further drilling purposes.

The gingival punch or mucosa punch shown in FIG. 1 is formed according to the state of the art, the recesses 9 also serve to enhance the preparation.

Due to one or more features of the trepan drill of the present disclosure and the possibility to select the inner and outer diameters accordingly based on the shape of the toothing and to adjust them exactly with respect to one another and to merge them smoothly, the already mentioned possibility to generate and implant a bone cylinder in a suitable manner is achieved.

Due to the alternating design of the cutting edges, the present disclosure makes sure that the generated chips do not get stuck in the toothing, but are discharged to the inside or outside.

The openings or recesses in the cylinder barrel of the surgical trepan drill are formed in a tangential direction with respect to the circumferential direction, such that no entrainment effect occurs when touching the soft tissue. Therewith, the danger of hurting the soft tissue is reduced.

Therewith, the trepans of the set are adjusted to one another and optimized in view of the combination of cutting width and shape congruency. Therewith, it is possible to create a transplant bed. By use of the first trepan drill, a circular cutout is produced in the bone material. By use of a second trepan drill, which is exactly guided by the centering tip (centering element 12), it is possible to create an adjacent circular groove. Therewith, a plane transplant bed surface can be created by using a plurality of stepped trepan drills. A remaining minor cylindrical stump in the center can be removed by a spoon. Therewith, there results a plane, annular transplant bed into which a bone cylinder, which was extracted from a different place by a trepan drill of the set, can be inserted accurately fittingly. Consequently, it is not required to use additional face-milling cutters or the like.

According to some embodiments of the present disclosure, a trepan drill may comprises a shaft having a clamping portion as well as a cylinder barrel shaped, hollow, core bit type head.

According to some embodiments of the present disclosure, it is provided that the toothing is made of teeth which are arranged in groups, which means that different teeth with different axial teeth height are alternatingly arranged in the circumferential direction. This means that individual groups are provided, respectively having different teeth or different arrangements of teeth. Therewith, the cutting characteristics of the trepan drill are enhanced. In particular, it is prevented that the drill runs uneasily and/or gets stuck in the bone material. Due to the arrangement of the individual teeth in some embodiments of the present disclosure in a group toothing, a smooth and efficient treatment is possible.

According to some embodiments of the present disclosure the trepan drill has a toothing which generates chips during its use, which chips can be collected and used later again for building up new bone tissue.

According to some embodiments of the present disclosure, a second tooth is arranged adjacent to the first tooth, said second tooth having a smaller axial tooth height than the first tooth having a larger tooth height (in the axial direction). Therewith, a small tooth follows a large tooth. In case the “large tooth” having a large tooth height gets stuck in the bone material, this does not influence the remaining toothing of the trepan drill. Rather, the subsequent smaller teeth effect a smoother, smaller cutting with reduced force. Therewith, the running of the complete trepan drill becomes smoother.

The cutting edges may provide alternating height, depth, and cutting angles, such that the generated chips do not accumulate, but are discharged either to the inside or the outside.

The head may comprise a group toothing including a first tooth as well as two further teeth, i.e. a second tooth and a third tooth. The first tooth is larger in the axial direction than the second or third tooth. Therewith, a plurality of group toothings of this kind result in the circumferential direction.

According to some embodiments of the present disclosure, it is particularly preferred (but not required) that a plurality of identically formed second teeth are arranged adjacent to one another. In such a group toothing, a leading first tooth is provided in the circumferential direction, and thus in the cutting direction, which is followed by at least two smaller, identically formed teeth.

In some embodiments of the present disclosure, the cutting edges of the teeth are arranged in mutually different axial arrangements at the distal end portion of the head. This means that the larger tooth axially protrudes more, while the second and further following smaller teeth are axially retreated. Even in case of a large tooth penetrating into the bone material, the trepan drill is thus supported and the further chipping process is continued by the smaller, axially retreated teeth.

The trepan drill may have a relatively wide tooth ring when viewed in the radial direction, such that the trepan drill may be used simultaneously for preparing a surgical implant bed.

According to some embodiments of the present disclosure, the teeth may be formed differently in view of their cutting edges and their cutting geometry. It is preferred (but not required) that the cutting angle lies in a range between about −20° to about +20°.

Also, the arrangement of the cutting edges can be varied in some embodiments of the present disclosure. It is possible to align the cutting edges of all teeth or of some teeth of the group teething radially with respect to the rotary axis of the trepan drill. However, it is also possible to arrange all cutting edges or only some of the cutting edges of the teeth of the group toothing at an angle with respect to the radial direction. The latter may enhance the flow of the chips and the removal of the bone chips.

In order to enhance the cutting characteristic and to minimize the thermal stress on the bone material due to friction at the cylinder part of the head, it may be preferred (but not required) in some embodiments of the present disclosure to provide the cylinder barrel of the core bit or of the head with recesses. These recesses may be formed round, elongate or in any other way. A plurality of rows of recesses may be provided in the axial direction. It may be advantageous if at least one recess in the circumferential direction includes a cutting edge such that also the outer periphery of the head may be cutting, in order to avoid a seizing of the bone material.

Due to the specific design of the openings as well as the curved transition region from working part to shaft, a use of the instruments preserving the soft tissue is possible using embodiments of the present disclosure.

The openings in the cylinder barrels may be formed in the tangential direction with respect to the rotary direction at the insert, such that they do not produce an entrainment effect when touching the soft tissue, such that the danger of hurting the soft tissue is reduced.

In some embodiments of the present disclosure, it is preferred (but not required) that the trepan drill is part of a set comprising at least two trepans. The trepan drill described above then serves to prepare a bone cylinder. A centering trepan to be used prior to the trepan drill serves for centering and for generating a first cut portion, into which the actual trepan drill is inserted subsequently.

The inner and outer diameters of the trepan drill of the set are configured such that they are exactly adjusted to each other and merge into each other smoothly, such that the obtained bone cylinder together with the trepan drill larger by one size exactly fits into the borehole which is generated by the trepan drill smaller by one size.

In addition, the toothing of some embodiments of the present disclosure enables a formation of a relatively wide tooth ring, such that it is possible to use the trepan drill in a system to produce an areal recess which is used as a transplant bed for placing a transplant in the subsequent preparation.

In some embodiments, the centering trepan also comprises a head supported on a shaft, a formed cylinder barrel shaped in the form of a core bit, and teeth with cutting edges at the distal end portion of the hollow cylinder of the core bit. In addition, a centering element is provided, which is preferably (but not always) formed integrally with the head. The one-part or integral form has the advantage that the centering trepan can be treated more easily (can be cleaned and disinfected more easily) and that the centering trepan shows less true running errors and therewith achieves a directed and smooth running.

All trepans of the set (trepan drill and centering trepan) as well as a possible gingival punch are preferably (but not always) made of a corrosion resistant steel, titan, or ceramics.

The centering trepan preferably (but not always) comprises a centering element which is formed as a helical drill, a triangle profile, a tip including a cutting edge (single cutter), or a wooden drill tip (front drill with centering tip).

In order to enhance the flow of chips and the removal of bone chips, it may be preferred (but not required) that the cutting edges of the centering trepan are arranged either in a radial direction or at an angle with respect to the radial direction also in the centering trepan.

In some embodiments, it is apparent that the various features of the trepan drill can be used in association with the gingival punch, and/or the centering trepan, or vice versa. For example, the configurations of teeth, cutting edges, and angles thereof shown in FIGS. 4-6, and/or any of the various recesses shown in FIGS. 7-10, may be used in association with a centering drill, such as the one depicted in FIG. 2. Further, the configurations of teeth, cutting edges, and angles thereof shown in FIGS. 11-12, or the recesses shown in FIG. 1 or 2, may be used in association with the trepan drill, such as the one depicted in FIG. 3.

We claim:

1. A trepan drill comprising a shaft including a clamping portion, and a head that comprises a substantially cylinder barrel shaped core bit and a distal end including a first, second, and third tooth that each respectively has a cutting edge, wherein the teeth each have different axial teeth heights and are arranged alternatingly in a circumferential direction.

2. The trepan drill of claim 1, wherein at least one of the second tooth and the third tooth is arranged adjacent to the first tooth and has an axial tooth height that is smaller than an axial tooth height of the first tooth.

3. The trepan drill of claim 2, wherein a plurality of identically formed second teeth and third teeth are arranged adjacent to one another.

4. The trepan drill of claim 1, wherein the cutting edges of the first, second, and third tooth are arranged at the distal end in different axial arrangements with respect to one another.

5. The trepan drill of claim 1, wherein at least one cutting edge is arranged in a radial direction.

6. The trepan drill of claim 1, wherein at least one cutting edge is arranged at an angle with respect to a radial direction.

7. The trepan drill of claim 1, wherein the surgical trepan drill is part of a set comprising at least two trepans.

8. The trepan drill of claim 7, wherein the set comprises a centering trepan comprising a shaft including a clamping portion, a centering element, and a head comprising a substantially cylinder barrel shaped core bit, wherein at least one tooth including a cutting edge is formed at a distal end portion of the core bit of the centering trepan.

9. The trepan drill of claim 8, wherein the centering element is formed integrally with the head.

10. The trepan drill of claim 8, wherein the centering element is formed in the shape of a helical drill.

11. The trepan drill of claim 8, wherein the centering element is formed in the shape of a triangle profile.

12. The trepan drill of claim 8, wherein the centering element is formed in the shape of a tip including a cutting edge.

13. The trepan drill of claim 8, wherein the centering element is formed in the shape of a front drill including a centering tip.

14. The trepan drill of claim 8, wherein the at least one cutting edge of the centering trepan is arranged in a radial direction of the centering trepan or at an angle with respect to the radial direction of the centering trepan.

15. A surgical trepan drill comprising a shaft including a clamping portion, and a head that comprises a substantially cylinder barrel shaped core bit and a distal end portion that includes a first, second, and third tooth, each tooth being arranged alternatingly in a circumferential direction and respectively having a cutting edge and an axial tooth height, wherein the first tooth is arranged in front of the second tooth and the third tooth in a cutting direction and formed larger in an axial direction than the second tooth and the third tooth, further wherein the second tooth and third tooth each follow the first tooth in the cutting direction and are formed identically.

16. The surgical trepan drill of claim 15, wherein the cutting edges of the second tooth and the third tooth are arranged identically in the axial direction.

17. The surgical trepan drill of claim 15, wherein the cutting edge of the first tooth is distally protruding further than the cutting edges of the second tooth and the third tooth.

18. The surgical trepan drill of claim 15, wherein the substantially cylinder barrel shaped core bit includes one or more recesses having an edge that is non-cutting in an external region.

19. The surgical trepan drill of claim 18, wherein at least one of the one or more recesses has a cutting edge in the circumferential direction.

20. The surgical trepan drill claim 18, wherein the one or more recesses are window-type recesses.

21. The surgical trepan drill of claim 15, further comprising a transitional area from the head to the shaft that extends convex-concave to preserve the soft tissue.

22. The surgical trepan drill of claim 15, further comprising a transitional area from the head to the shaft that extends without a sharp edge to preserve the soft tissue.

23. The surgical trepan drill of claim 15, wherein at least one tooth has a cutting angle in the range of between about −20° to about +20°.
